(19) United States
(12) Patent Application Publication
(10) Pub. No.: US 2023/0348913 A1
(43) Pub. Date: Nov. 2, 2023

(54) METHODS TO RE-ENGAGE A FETAL WOUND HEALING PATHWAY FOR ADULT SKIN REPAIR

(71) Applicant: THE TRUSTEES OF INDIANA UNIVERSITY, Bloomington, IN (US)

(72) Inventors: Chandan K. SEN, Indianapolis, IN (US); Subhadip GHATAK, Indianapolis, IN (US); Sashwati ROY, Indianapolis, IN (US)

(21) Appl. No.: 17/800,680

(22) PCT Filed: Mar. 2, 2021
(86) PCT No.: PCT/US2021/020373

§ 371 Date: Aug. 18, 2022

Related U.S. Application Data
(60) Provisional application No. 62/985,008, filed on Mar. 4, 2020.

(51) Int. Cl. C12N 15/113; C12N 15/86; A61P 17/02

(52) U.S. Cl. CPC C12N 15/1137; C12N 15/86; A61P 17/02; C12N 2310/113; C12N 2310/3231; C12N 2740/15043; C12N 2740/15071

(57) ABSTRACT

Compositions and methods are provided for promoting wound healing in a subject by administering a composition that enhances the expression of the protein nonselenocysteine-containing phospholipid hydroperoxide glutathione peroxidase (NPGPx) in wound-edge tissues. In accordance with one embodiment such compositions are used in conjunction with known treatments for use on chronic wounds including in diabetic patients.

### CROSS REFERENCE TO RELATED APPLICATIONS

This application claims priority to U.S. Provisional Patent Application No. 62/985,008 filed on Mar. 4, 2020, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 4 kilobytes ACII (Text) file named “333982_ST25.txt,” created on March 1, 2021.

### BACKGROUND OF THE DISCLOSURE

Nonhealing chronic wounds are a challenge to the patient, the health care professional, and the health care system. They significantly impair the quality of life for millions of people and impart a burden on society in terms of lost productivity and health care dollars.

Fetal wound healing is more efficient and regenerative than adult wound healing. Fetal skin tissue perfectly executes epidermal regeneration; a pattern that is absent during adult wound healing. Prior to the present disclosure it was unknown if the drivers of the fetal repair process continued to exist in adult tissue, and if so, whether the critical elements of the fetal repair process could be activated in adult tissue to improve adult tissue repair outcomes.

The protein nonselenocysteine-containing phospholipid hydroperoxide glutathione peroxidase (NPGPx) is an oxidant stress sensor protein. NPGPx is abundantly expressed in normal fetal epidermis, but is not expressed adult epidermis. NPGPx is a direct target of the miR-29 family and is variably induced upon adult tissue wounding. More particularly, after injury, the abundance of miR-29 is lowered, and this lower abundance of miR-29 after injury permits NPGPx transcripts and protein expression to promptly increase after injury in adult wound-edge tissue.

The low abundance of miR-29 after injury is due to pre-miRNAs being rapidly degraded by the protein endoribonuclease monocyte chemoattractant protein-induced protein 1 (MCPIP1). MCPIP1 is induced at the site of the wound by lipids that are blood-clot derived post-injury. This favorable physiologic response was blunted in non-healing wounds, such as in diabetic mice.

This pathway may be schematically illustrated as:

MCPIP1→↓miR-29→↑NPGP_{X}.

The MCPIP1→↓miR-29→↑NPGP_{X} pathway induced more regenerative healing in adult wounds by re-engaging expression of numerous developmentally active coding genes and fetal proteins relevant for tissue formation and repair.

Accordingly, there is a need for treatments that enhance would repair by stimulating the fetal wound healing pathway in adult skin repair.

### SUMMARY

In accordance with one embodiment of the present disclosure, a method of promoting wound healing in a subject is provided, the method comprising the step of administering a composition that enhances the expression of the protein nonselenocysteine-containing phospholipid hydroperoxide glutathione peroxidase (NPGPx) in wound-edge tissues. In one embodiment the composition comprises an anti-miR-29 oligonucleotide and a pharmaceutically acceptable carrier, wherein the composition is formulated for introduction into the cytosol of wound edge tissues. In one embodiment the anti-miR-29 oligonucleotides are provided in a carrier such as a viral vector or lipid vessel. In one embodiment the composition is formulated for transmission by electroporation. In accordance with one embodiment, compositions for enhancing NPGPx concentrations in wound-edge tissues as disclosed herein are used in conjunction with known treatments for use on chronic wounds including in diabetic patients.

In one embodiment a method to regulate wound healing in adult skin, optionally the adult skin of a diabetic individual, is provided. In one embodiment the method enhances regenerative healing in an adult wound by re-engaging expression of developmentally active coding genes and fetal proteins in tissue formation and repair. In one embodiment the method comprises augmenting physiologic expression of the protein nonselenocysteine-containing phospholipid hydroperoxide glutathione peroxidase (NPGPx) in wound-edge tissues. In one embodiment, increased expression of NPGPx is induced in wound-edge tissues by transfecting the cells of wound-edge tissues with nucleic acid sequences that stimulate increased expression of NPGPx, optionally by suppressing miR-29, resulting in regulated wound healing in adult skin. In one embodiment accelerated wound healing in adult skin is promoted by manipulating at least one component of the following pathway: by increasing endoribonuclease monocyte chemoattractant protein-induced protein (MCPIP1), decreasing miR-29, reducing levels of miR-29 activity, and increasing the cellular concentration of nonselenocysteine-containing phospholipid hydroperoxide glutathione peroxidase (NPGPx), resulting in accelerated wound healing and wound closure of wounds in adult skin.

In one embodiment the method facilitates healing of a wound in a diabetic patient by augmenting expression of nonselenocysteine-containing phospholipid hydroperoxide glutathione peroxidase (NPGPx) expression at the wound site. In one embodiment the wound is a non-healing/chronic wound. In one embodiment the method increases the expression of NPGPx and this increased expression of NPGPx overcomes a deleterious effect of diabetes on wound closure.

In one embodiment the method comprises topical delivery, to a patient in need thereof, of anti-miR-29 oligonucleotides by topical tissue nanotransfection (TNT) to directly induce nonselenocysteine-containing phospholipid hydroperoxide glutathione peroxidase (NPGPx) in skin keratinocytes.

In one embodiment a method to regulate wound healing in adults is provided wherein the method comprises topically delivering anti-miR-29 oligonucleotides by topical tissue nanotransfection (TNT) under conditions sufficient to induce nonselenocysteine-containing phospholipid hydroperoxide glutathione peroxidase (NPGPx) in skin keratinocytes and result in wound healing.

In one embodiment a pharmaceutical composition for enhancing wound closure is provided, wherein said composition comprises an oligonucleotide at least 6, 7, or 8 nucleotides in length, wherein the oligonucleotide has at least 85% sequence identity to a continuous 6-8 nucleotide sequence of a human mature miR-29 sequence selected from the group consisting of SEQ ID NO: 2, SEQ ID NO: 3, SEQ ID NO: 4, SEQ ID NO: 5, SEQ ID NO: 6 and SEQ ID NO: 10, or a complement of any of those sequences thereof and a pharmaceutically acceptable carrier.

### BRIEF DESCRIPTION OF THE DRAWINGS

FIGS. 1A-1H. FIG. 1A shows miR-29a, miR-29b and miR-29c transcript abundance in skin and wound-edge tissue of adult C57BL/6 mice at different time points post-wounding (PW) (n=4). FIG. 1B shows the binding of positions 41-47 of mNPGPx 3′ UTR (SEQ ID NO: 1) to mmu-miR-29a-3p (SEQ ID NO: 2), mmu-miR-29b-3p (SEQ ID NO: 3) and mmu-miR-29a-3p (SEQ ID NO: 4). FIG. 1C shows NPGPx transcript abundance in skin and wound-edge tissue in adult C57BL/6 mice (n=6). FIG. 1D shows Western blot analysis of NPGPx in skin and wound-edge tissue in adult C57BL/6 mice; GAPDH was the loading control and independent blots were repeated at least three times with similar results. FIG. 1E shows NPGPx transcript abundance in laser captured microdissected (LCM) keratinocytes from skin and day 7 wound-edge tissue of C57BL/6 mice (n=4). FIG. 1F shows Western blot analysis of NPGPx in murine fetal skin (E15.5-E18.5) and adult skin; GAPDH was the loading control and independent blots were repeated at least three times with similar results. FIG. 1G shows NPGPx transcript abundance in human fetal and adult skin (n=6, 5). FIG. 1H shows Western blot analysis of NPGPx in human fetal and adult skin; each lane indicates separate biological samples (n=5) and GAPDH was the loading control. All data were shown as mean±SEM.

FIGS. 2A-2F show the role of NPGPx for fetal and adult wound closure. The NPGPx expressed in human epidermis correlated with the highest level of wound closure (FIG. 2A). NPGPx suppression impaired adult wound closure (FIGS. 2B and 2D). NPGPx overexpression improved adult wound closure (FIGS. 2C and 2E). NPGPx overexpression accelerated wound re-epithelialization with significant improvement in skin barrier function (FIG. 2F); a critical functional test of re-epithelialization.

FIGS. 3A-3K show NPGPx in diabetic db/db mice with mutations of the leptin receptor that display impaired wound healing and serve as a model for non-healing wounds. FIGS. 3A and 3B show miR-29a, miR-29b and miR-29c expression in fetal (E15.5-E-18.5) and adult skin of C57BL/6 mice (FIG. 3A), and fetal and adult human skin (FIG. 3B). (FIG. 3A: n=5; FIG. 3B: n=6, 6; 6,6; 5,6). FIG. 3C shows NPGPx transcript abundance in skin and wound-edge tissue in K14-dicer+/+ and K14-dicer−/− mice (n=4). FIGS. 3D and 3E show Western blot analysis of HaCaT cells transfected with miR-29b or miR-29c inhibitor (FIG. 3D) and miR-29b or miR-29c mimic (FIG. 3E). GAPDH was the loading control. Independent blots were repeated at least three times with similar results. FIGS. 3F and 3G show miRNA target reporter luciferase assay in in HaCaT cells after delivery of miR-29b (FIG. 3F) and miR-29c (FIG. 3G) mimic (H: n=6; I: n=6). FIG. 3H shows NPGPx transcript abundance in skin and day 7 wound-edge tissue of non-diabetic (m+/db, littermate control) and diabetic db/db mice (n=4). Western blot analysis of NPGPx from skin and day 7 wound-edge tissue from m+/db and db/db mice showed similar results as obtained for transcript abundance. Independent blots were repeated at least three times with similar results. FIG. 3I shows miR-29a, miR-29b and miR-29c expression in day 7 wound-edge tissue in m+/db and db/db mice (n=4). Fold change was calculated using respective skin (m+/db and db/db) as 1. FIG. 3J shows NPGPx transcript abundance of NPGPx in day wound-edge tissue of diabetic db/db mice treated with either control (pLVcon) or NPGPx over expressing (pLVNPGPx) lentivirus. Western blot analysis mice showed similar results as obtained for transcript abundance. GAPDH was used as loading control. Digital photographs of wounds in db/db mice were taken at day 0, 6 and 10 and wound closure was presented as percentage of initial wound area. (n=4) in FIG. 3K.

FIGS. 4A-4F shows the role of each of MCPIP1, miR-29, and NPGPx in increased wound healing. Suppression of miR-29b and miR-29c significantly elevated NPGPx expression (FIGS. 4A and 4B). Delivery of miR-29b and miR-29c mimics significantly suppressed both NPGPx expression (FIG. 4B), as well as NPGPx 3′-UTR reporter luciferase activity. FIG. 4C gives the sequence of wild-type NPGPx 3′-UTR (top; SEQ ID NO: 8) and NPGPx 3′-UTR (SEQ ID NO: 9) with the mutation of predicted binding site (seed region) cloned in the reporter construct (bottom).

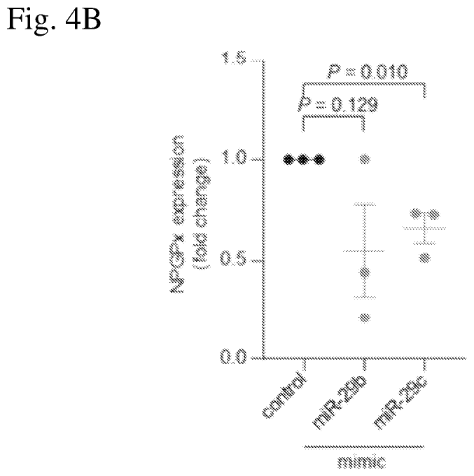

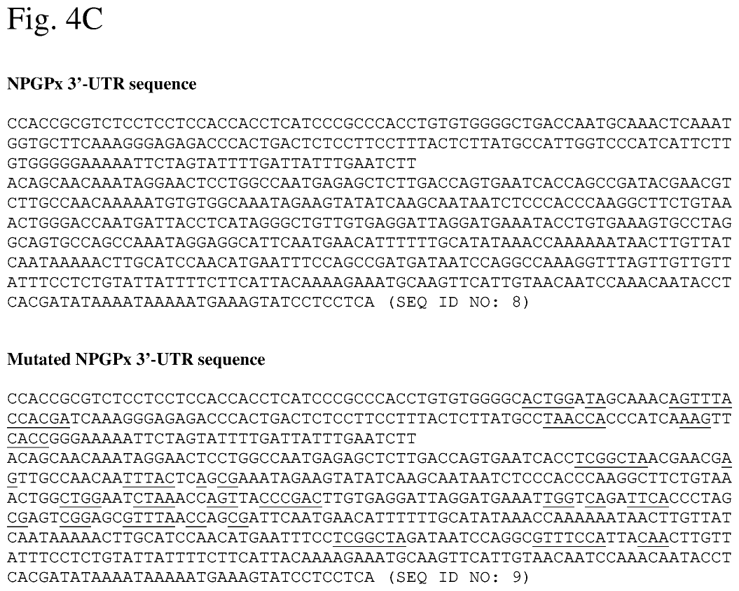

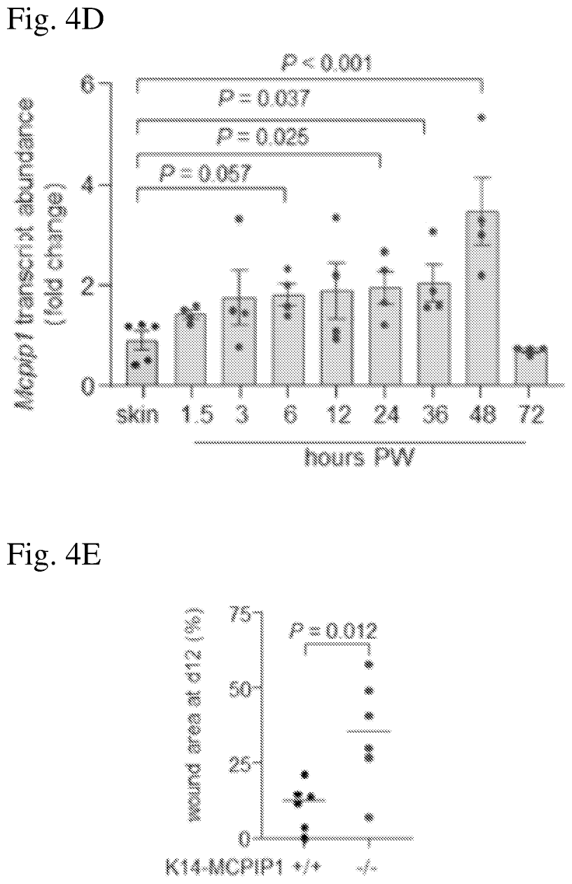

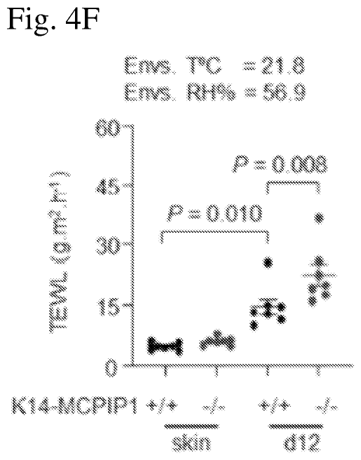

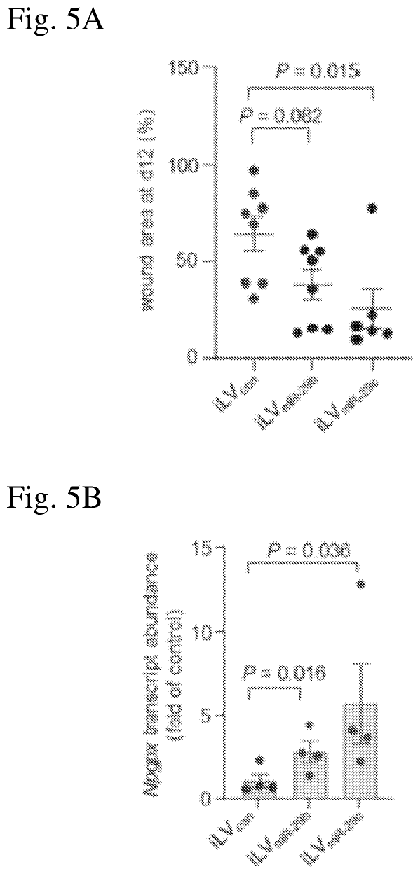

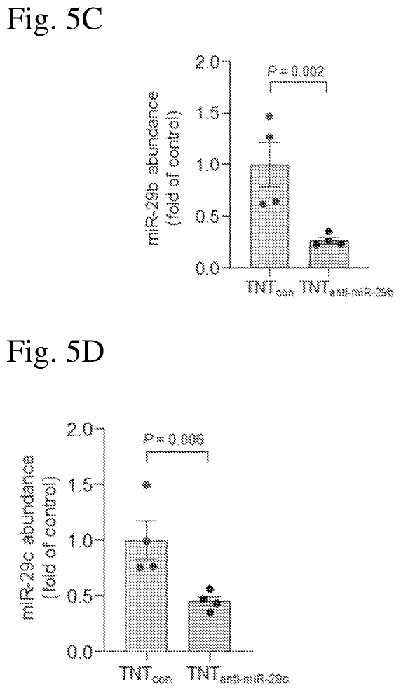

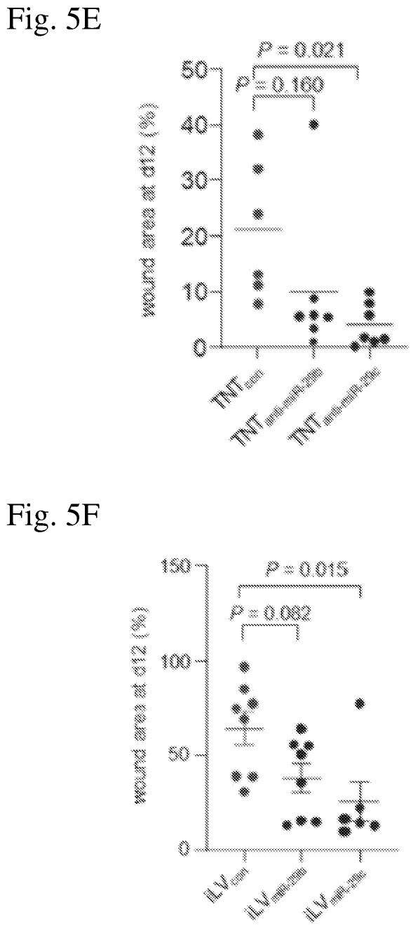

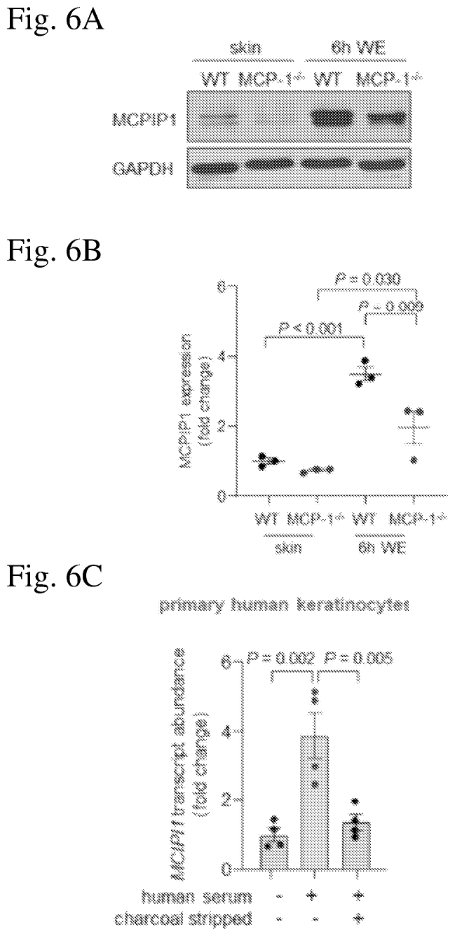

Positions mutated were marked with underlined text. Mutations of the predicted binding sites (seed sequences) in the 3′-UTR of NPGPx (FIG. 4C) abolished miR-29b and miR-29c dependent translational repression. FIG. 4D shows MCPIP1 transcript abundance in skin and wound-edge tissue at different time points PW in adult C57BL/6 mice, (n=5, 4). Digital photographs of wounds in K14-MCPIP1^{+/+} and K14-MCPIP1^{-/-} mice were taken and wound closure presented as percentage of initial wound area (n=6); scale, 2 mm (FIG. 4E). FIG. 4F shows transepidermal water loss (TEWL) at d12 post-wounding is lower in K14-MCPIP1^{+/+} and higher in K14-MCPIP1^{-/-} mice suggesting poor barrier function, (n=6).

FIGS. 5A-5F show use of topical tissue nanotransfection (TNT) chip 2.0 to deliver agents to demonstrate increased NPGPx and decreased miR-29b and miR-29c result in increased wound healing. Wound closure occurred significantly more quickly (FIG. 5A) by induction of NPGPx expression in the skin following miR-29b and miR-29c suppression (FIG. 5B). FIGS. 5C and 5D show expression of miR-29b (FIG. 5C) and miR-29c (FIG. 5D) in laser-captured epidermis of C57BL/6 mice 24 h post-TNT of LNA-control, LNA-anti-miR-29b, and LNA-anti-miR-29c mice (n=4). FIG. 5E presents wound closure data (FIG. 5F) at day 12 wounds in K14-MCPIP1^{-/-} mice based on percent wound closure, showing accelerated re-epithelialization in wounds treated with LNA-anti-miR-29b, and LNA-anti-miR-29c relative to LNA-control. All data were shown as mean±SEM.

FIGS. 6A-6C provide data showing that MCPIP1 plays a role in adult wound healing and that MCPIP1 is induced post-wounding independent of MCP1. FIG. 6A shows Western blot analysis and FIG. 6B shows quantification of MCPIP1 from skin and wound-edge tissue collected at 6 h post-wounding (PW) from wild type (WT) and MCP-1 knockout mice. GAPDH was a loading control. Independent blots were repeated at least three times with similar results. Data are expressed as mean±SEM (n=3). FIG. 6C shows the role of serum in the induction of MCPIP1 and the expression of MCPIP1 in primary human keratinocytes after removal of bioactive phospholipids from serum by charcoal stripping method (n=4). As shown by the data, the presence of a blood clot and MCP-1 increases cellular concentrations of MCPIP1.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein an exogenous sequence in reference to a cell is a sequence that has been introduced into the cell from a source external to the cell.

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5× SSC (150 mM NaCl, 15 mM trisodium citrate), 50 mM sodium phosphate (pH 7.6), 5× Denhardt's solution, 10% dextran sulfate, and 20 μg/ml denatured, sheared carrier DNA such as salmon sperm DNA, followed by washing the hybridization support in 0.1× SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine (hCys)

V. Large, aromatic residues:
Phe, Tyr, Trp, acetyl phenylalanine, napthylalanine (Nal)

As used herein the term “patient” without further designation is intended to encompass any warm blooded vertebrate domesticated animal (including for example, but not limited to livestock, horses, cats, dogs and other pets) and humans and includes individuals not under the direct care of a physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini.

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M , methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g. , BrdU), and non-phosphodiester internucleoside linkages (e.g. , peptide nucleic acid (PNA) or thiodiester linkages) . In particular, nucleic acids can include, without limitation, DNA, RNA, cDNA, gDNA, ssDNA, dsDNA or any combination thereof.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C) , guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein “Interfering RNA” is any RNA involved in post-transcriptional gene silencing, which definition includes, but is not limited to, double stranded RNA (dsRNA), small interfering RNA (siRNA), and microRNA (miRNA) that are comprised of sense and antisense strands.

As used herein a “locked nucleic acid” (LNA), is a modified RNA nucleotide in which the ribose moiety is modified with an extra bridge connecting the 2′ oxygen and 4′ carbon. For example, a locked nucleic acid sequence comprises a nucleotide of the structure:

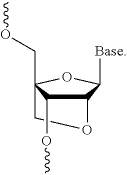

As used herein the term “vasculogenesis” is defined as the differentiation of precursor cells (angioblasts) into endothelial cells and the de novo formation of a primitive vascular network.

As defined herein “wound healing” defines a process wherein a living organism replaces destroyed or damaged tissue by newly produced tissue. The process includes three phases blood clotting, tissue growth (cell proliferation), and tissue remodeling. Accelerated wound healing includes a shorten length of time required to complete any of three phases, including for example the closure of an open wound due to tissue growth.

As disclosed herein a generic reference to miR-29 is intended to include all known variants of mammalian miR-29 including for example human mature forms miR-29a, miR-29b and miR-29c of SEQ ID NO: 2, SEQ ID NO: 3 and SEQ ID NO: 4, respectively.

### EMBODIMENTS

Major differences exist in the processes of fetal tissue healing and adult tissue healing, including different pathways, growth factors, cytokines, interleukins, matrix metalloproteinases, extracellular matrix molecules (ECM), inflammatory cells, and cell surface molecules utilized in the two responses. Fetal repair processes engage specific combinations of these pathways to permit efficient restitution of all cellular elements and appendages (hair follicles and sweat glands) upon skin wounding, with appropriate remodeled ECM components to provide proper developmental barrier and biomechanical properties. Most of these regenerative processes are extinguished prior to birth. As disclosed herein methods are provided for activating components of the fetal wound healing process in adult cell on a temporary basis to enhance and/or accelerate wound healing, including wound closure.

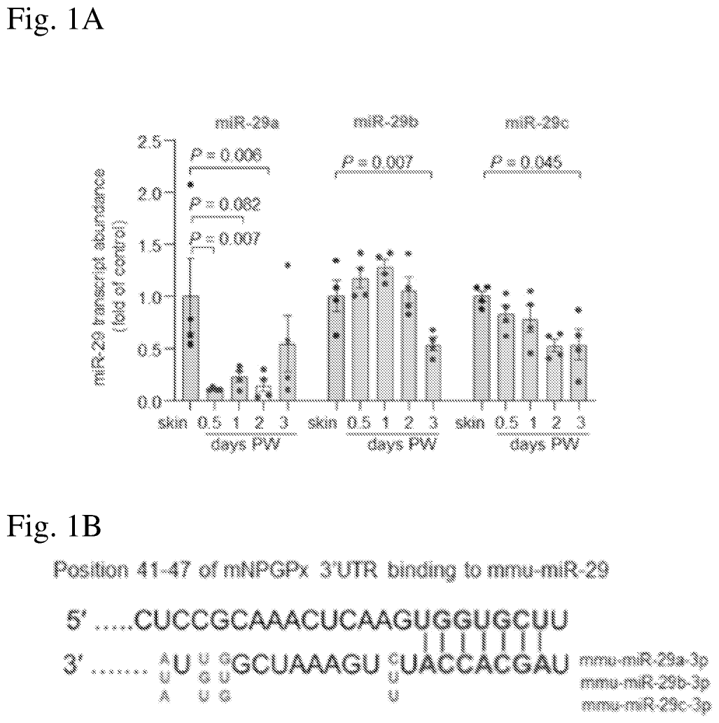

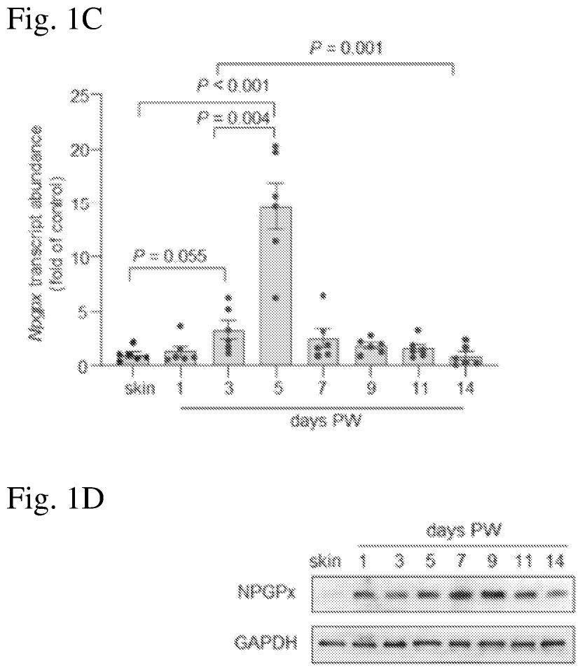

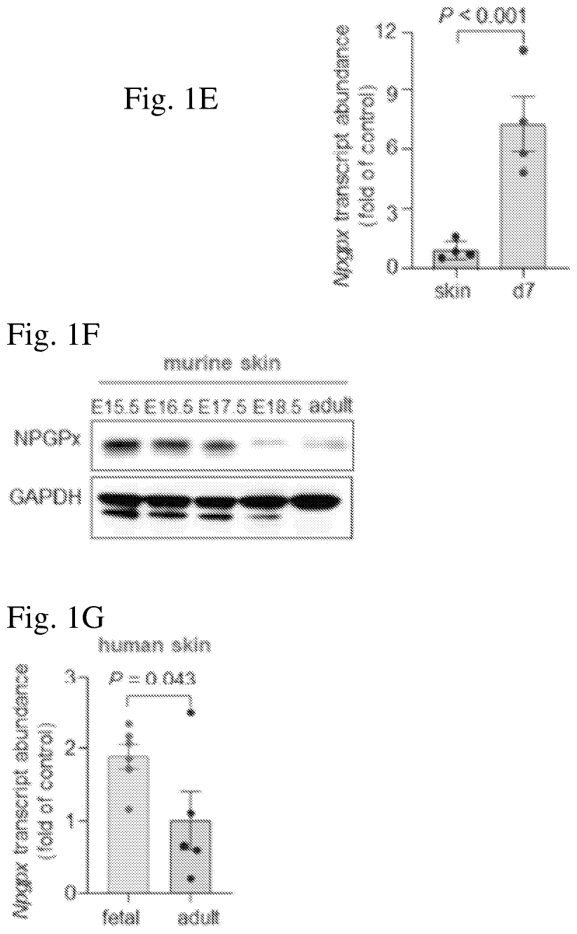

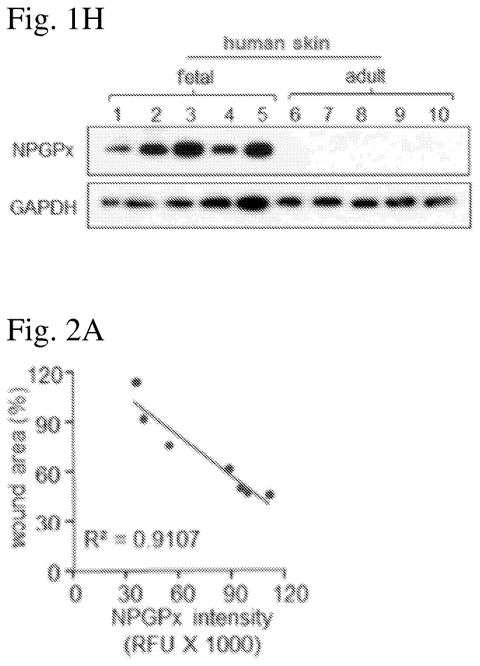

Numerous microRNAs (miRNAs) are temporally and spatially muted in fetal tissues, presumably to enable fetal tissue to execute rapid developmental processes. While this difference is a key contrast between fetal tissue and adult tissue, it is poorly understood. For example, low abundance of the miR-29 family during fetal development has been reported in skin across several species. After acute skin injury, the miR-29 family is suppressed over the first 7 days commencing within 48-72 h of the perturbation (FIG. 1A).

The miR-29 family is predicted to have conserved binding sites on the 3′-untranslated regions (3′-UTRs) of twenty collagen genes independent of sequence homology. In adult tissue, overexpression of the miR-29 family members suppresses ECM genes, resulting in abnormal tissue repair. In silico analyses predicted that, in addition to these known ECM proteins, miR-29 might contain potential binding site(s) for the 3′-UTRs (FIG. 1B) of the oxygen stress sensor protein nonselenocysteine-containing phospholipid hydroperoxide glutathione peroxidase (NPGPx), that is conserved among all vertebrates. Applicant have demonstrated NPGPx is wound-inducible as a response to the reactive oxidant stress, based on work demonstrating the key role of injury-induced generation of NOX-dependent reactive oxygen species at the skin wound-edge. Alterations in NPGPx expression cause systemic evidence of excessive oxidative stress, cardiovascular disease, obesity, autoimmunity, increased risk for carcinogenesis, and shortened lifespan in mice. However, the biological significance of NPGPx as a key element in tissue injury and repair remains an enigma because a critical selenocysteine residue at its catalytic center is absent, rendering NPGPx catalytically inactive as a GPx.

In accordance with one embodiment a method of accelerating wound healing and/or wound closure in adult skin of a subject is provided. In one embodiment the wound is a chronic or non-healing wound, optionally wherein the patient is a diabetic. The method comprising the step of increasing the concentration of the protein nonselenocysteine-containing phospholipid hydroperoxide glutathione peroxidase (NPGPx) in the cells of wound-edge tissue. In accordance with one embodiment the cells of wound-edge tissue are transfected with nucleic acid sequences, using any transfection technique known to the skilled practitioner, that result in increased cellular concentration of NPGPx. The nucleic acid sequences introduced into the cells can be gene encoding sequences or interference oligonucleotides. Increased expression of NPGPx, as demonstrated herein can be accomplished by increasing cellular concentrations of endoribonuclease monocyte chemoattractant protein-induced protein (MCPIP1) or Monocyte chemoattractant protein-1 (MCP-1) or by decreasing active miR-29 cellular concentrations, including reducing miR-29b or miR-29c cellular concentrations.

In one embodiment a method of enhancing or accelerating wound healing is provided wherein wound-edge tissue is transfected with a modifier of miR-29 activity in an amount effective to lower miR-29 activity and increase NPGPx expression. In one embodiment the modifier the of miR-29 activity is a gene encoding the protein endoribonuclease monocyte chemoattractant protein-induced protein 1 (MCPIP1). In one embodiment the modifier of miR-29 activity is an oligonucleotide at least 6, 7, 8, 9 or 10 nucleotides in length, wherein the oligonucleotide has at least 85%, 90%, 95%, 99% sequence identity to a continuous nucleotide complementary sequence of SEQ ID NO: 10. In one embodiment the modifier of miR-29 activity is an oligonucleotide at least 6, 7, 8, 9 or 10 nucleotides in length, wherein the oligonucleotide has at least 85%, 90%, 95%, 99% sequence identity to a continuous nucleotide sequence of SEQ ID NO: 11. In one embodiment the modifier of miR-29 activity is an oligonucleotide at least 6, 7, 8, 9 or 10 nucleotides in length, wherein the oligonucleotide has 100% sequence identity to a continuous nucleotide sequence of SEQ ID NO: 11. In one embodiment the modifier of miR-29 activity is an oligonucleotide at least 8 nucleotides in length, wherein the oligonucleotide has at least 85% sequence identity to a continuous 8 nucleotide sequence of human mature miR-29a (UAGCACCAUCUGAAAUCGGUUA SEQ ID NO: 2) or a complement thereof. In one embodiment the modifier of miR-29 activity is an oligonucleotide at least 8 nucleotides in length, wherein the oligonucleotide has at least 85% sequence identity to a continuous 8 nucleotide sequence of human mature miR-29b (UAGCACCAUUUGAAAUCAUGUU; SEQ ID NO: 3) or miR-29c (UAGCACCAUUUGAAAUCGGUUA; SEQ ID NO: 4 or a complements thereof. In one embodiment the modifier of miR-29 activity comprises an oligonucleotide comprising SEQ ID NO: 5 or SEQ ID NO: 6.

In one embodiment the interference RNA comprises a locked nucleic acid. In one embodiment the locked nucleic acid is located at i) the N-terminus; ii) the C-terminus; or iii) at both the N-terminus and the C-terminus of the oligonucleotide.

In one embodiment an anti-miR-29 oligonucleotide is directed for delivery into the cytosol of human keratinocyte cells. In one embodiment the oligonucleotide is delivered into the cytosol of cells via skin electroporation or tissue nanotransfection. In one embodiment the oligonucleotide is delivered into the cytosol of cells via a viral vector.

In accordance with one embodiment, a method is provided for promoting wound healing in a subject by administering a therapeutic agent that reduces miR-29 activity. In one embodiment a miR-29 inhibitor is brought in contact with a wound on subject, in an amount effective to reduce the function or activity of miR-29, thereby promoting wound healing. In one embodiment miR-29 inhibitor is delivered locally to the wound by physical contact of a topical formulation, or by injection of an miR-29 inhibitor into wound-edge tissue. In one embodiment, the miR-29 inhibitor is administered by skin electroporation or tissue nanotransfection. In one embodiment, the miR-29 inhibitor is an oligonucleotide, including for example an oligonucleotide comprising a locked nucleic acid (LNA) conjugated antisense miR-29 oligonucleotide, optionally wherein the antisense miR-29 oligonucleotide is at least 6 nucleotides in length and shares at least 95, 99 or 100% sequence identity with a sequence selected from the group consisting of SEQ ID NO: 2, SEQ ID NO: 3, SEQ ID NO: 4, SEQ ID NO: 5, SEQ ID NO: 6 and SEQ ID NO: 10, or a complement of any of those sequences.

The miR-29 inhibitor oligonucleotides disclosed herein may comprise one or more locked nucleic acid (LNAs) residues, or “locked nucleotides.” The oligonucleotides of the present invention may comprise one or more nucleotides containing other sugar or base modifications. The terms “locked nucleotide,” “locked nucleic acid unit,” “locked nucleic acid residue,” “LNA” or “LNA unit” may be used interchangeably throughout the disclosure and refer to a bicyclic nucleoside analogue. For instance, suitable oligonucleotide inhibitors can be comprised of one or more “conformationally constrained” or bicyclic sugar nucleoside modifications (BSN) that confer enhanced stability to complexes formed between the oligonucleotide containing BSN and their complementary target strand.

In one embodiment the miR-29 inhibitory oligonucleotide may comprise, consist essentially of, or consist of, an interference RNA or antisense sequence to miR-29a (SEQ ID NO: 2), miR-29b (SEQ ID NO: 2), or miR-29c (SEQ ID NO: 4). In one embodiment, the oligonucleotide comprises an antisense sequence directed to miR-29b or miR29c. For example, the oligonucleotide can comprise a sequence of at least 8 nucleotides that has at least about 75%, 76%, 77%, 78%, 79%, 80%, 81%, 82%, 83%, 84%, 85%, 86%, 87%, 88%, 89%, 90%, 91%, 92%, 93%, 94%, 95%, 15 96%, 97%, 98%, or 99% sequence identity to a continuous 8 nucleotide sequence of human mature miR-29a (SEQ ID NO: 2), miR-29b (SEQ ID NO: 3) or miR-29c (SEQ ID NO: 4). In one embodiment the miR-29 inhibitor is an oligonucleotide at least 6, 7, or 8 nucleotides in length, wherein the 6, 7, or 8 nucleotides of the oligonucleotide has 100% sequence identity to a continuous 6, 7 or 8 nucleotide sequence of human miR-29a sequence (SEQ ID NO: 2), miR-29b (SEQ ID NO: 3) or miR-29c (SEQ ID NO: 4) or any complement of those sequences. In one embodiment, the oligonucleotide inhibitor as provided herein comprises a sequence that has at least 95% sequence identity to SEQ ID NO: 5 or SEQ ID NO: 6. In one embodiment, the oligonucleotide inhibitor as provided herein comprises a sequence that has 100% sequence identity (i.e., fully complementary) with a contiguous sequence found within the mature miR-29a, miR-29b, or miR-29c sequence, or a complement thereof. It is understood that the sequence of the oligonucleotide inhibitor is considered to be complementary to miR-29a, miR-29b, or miR-29c even if the oligonucleotide inhibitor sequence includes a modified nucleotide instead of a naturally-occurring nucleotide.

In one embodiment the oligonucleotide miR-29 inhibitor is an RNA 6-15 nucleotide in length and comprising a sequence that has at least 80, 85, 90, 95 or 99% sequence identity with a contiguous sequence found in an miR-29a (SEQ ID NO: 2), miR-29b (SEQ ID NO: 3), or miR-29c (SEQ ID NO: 4) sequence or a complement thereof, respectively. In one embodiment the oligonucleotide miR-29 inhibitor is an RNA comprising the sequence of SEQ ID NO: 5 or SEQ ID NO: 6, or a complement thereof, or the corresponding DNA or its complement. In one embodiment any of the oligonucleotide miR-29 inhibitors disclosed herein further comprises a locked nucleic acid. In one embodiment the oligonucleotide comprises two or more locked nucleic acids. In one embodiment the oligonucleotide miR-29 inhibitor is an RNA comprising

i) a single locked nucleic acid at its 5′ terminus;

ii) a single locked nucleic acid at its 3′ terminus; or

iii) a locked nucleic acid at its 5′ and 3′ terminus.

In one embodiment the oligonucleotide miR-29 inhibitor is an RNA comprising the sequence of SEQ ID NO: 5 or SEQ ID NO: 6 and an additional locked nucleic acid, located at its 5′ terminus or 3′ terminus or at both the 5′ terminus and the 3′ terminus.

The wound to be treated in accordance with the present disclosure may be a surgical wound, a chronic wound, or an acute wound. In addition, the wound may be an incision, a pressure ulcer, a venous ulcer, an arterial ulcer, a diabetic lower extremity ulcer, a laceration, an abrasion, a puncture, a contusion, an avulsion, a cavity, a burns, or any combination thereof. The wound may be a wound edge, a wound bed, and/or a peri-wound.

In one embodiment, a method of promoting wound healing in a subject comprises administering to the subject a miR-29 inhibitor, such as an oligonucleotide disclosed herein. In some embodiments, the subject suffers from diabetes. In some embodiments, healing of a chronic wound, diabetic foot ulcer, venous stasis leg ulcer or pressure sore is promoted by administration of a miR-29 inhibitor. In one embodiment the method comprises transfecting the cells of wound-edge tissue with one or more oligonucleotides having a length of at least 6, 7 or 8 nucleotides, wherein the oligonucleotides are selected from oligonucleotides having at least 80%, 85, 90%, 95% or 99% sequence identity to a continuous 6, 7 or 8 nucleotide sequence of human mature miR-29a sequence (SEQ ID NO: 2), miR-29b (SEQ ID NO: 3), miR-29c sequence (SEQ ID NO: 4), SEQ ID NO: 5, SEQ ID NO: 6 or any complement of said sequences.

In one embodiment, administration of a miR-29 inhibitor as provided herein provides at least about 5%, 10%, 20%, 30%, 40%, 50%, 60%, 70%, 80%, or 90% improvement in wound re-epithelialization or wound closure as compared to a wound not administered the miR-29 inhibitor relative to time. In some embodiments, administration of a miR-29 inhibitor as provided herein provides at least about 5%, 10%, 20%, 30%, 40%, 50%, 60%, 70%, 80%, or 90% more granulation tissue formation or neovascularization as compared to a wound not administered the miR-29 inhibitor.

In one embodiment, administration of a miR-29 inhibitor as provided herein provides at least about 5%, 10%, 20%, 30%, 40%, 50%, 60%, 70%, 80%, or 90% improvement in wound re-epithelialization or wound closure as compared to a wound administered an agent known in the art for treating wounds relative to time. In some embodiments, administration of a miR-29 inhibitor as provided herein provides at least about 5%, 10%, 20%, 30%, 40%, 50%, 60%, 70%, 80%, or 90% more granulation tissue formation or neovascularization as compared to a wound administered an agent known in the art for treating wounds relative to time.

In accordance with the present invention nucleic acids and/or proteins are introduced into the cytosol of cells of wound-edge tissue, including for example dermal fibroblasts or keratinocytes, to decrease the concentration of function miR-29 in the target cells. Any of the standard techniques for introducing macromolecules into cells can be used in accordance with the present disclosure. Known delivery methods can be broadly classified into two types. In the first type, a membrane-disruption-based method involving mechanical, thermal or electrical means can be used to disrupt the continuity of the cell membrane with enhanced permeabilization for direct penetration of desired macromolecules. In the second type, a carrier-based method, using various viruses, exosomes, vesicles and nanoparticle capsules, allows uptake of the carrier through endocytosis and fusion processes of cells for delivery of the carrier payload.

In one embodiment intracellular delivery is via a viral vector, or other delivery vehicle capable of interacting with a cell membrane to deliver its contents into a cell. In one embodiment intracellular delivery is via three-dimensional nanochannel electroporation, delivery by a tissue nanotransfection device, or delivery by a deep-topical tissue nanoelectroinjection device. In one embodiment the miR-29 inhibitor is delivered into the cytosol of cells of wound-edge tissues in vivo through tissue nanotransfection (TNT) using a silicon hollow needle array.

Among the methods of permeabilization-based disruption delivery, electroporation has already been established as a universal tool. High efficiency delivery can be achieved with minimum cell toxicity by careful control of the electric field distribution. In accordance with one embodiment nucleic acid sequences are delivered to the cytosol of somatic cells through the use of tissue nanotransfection (TNT). Tissue nanotransfection (TNT) is an electromotive gene transfer technology that delivers plasmids, RNA and oligonucleotides to live tissue causing direct conversion of tissue function in vivo under immune surveillance without the need for any laboratory procedures. Unlike viral gene transfer commonly used for in vivo tissue reprogramming, TNT obviates the need for a viral vector and thus minimizes the risk of genomic integration or cell transformation.

Current methods can involve transfecting cells in vivo or in vitro followed by implantation. Although one embodiment of the present invention entails in vitro transfection of cells followed by transplantation, cell implants are often met with low survival and poor tissue integration. Additionally, transfecting cells in vitro involves additional regulatory and laboratory hurdles.

In accordance with one embodiment the cells of wound-edge tissue are transfected in vivo with an miR-29 interference oligonucleotide comprising composition as disclosed herein. Common methods for bulk in vivo transfection are delivery of viral vectors or electroporation. Although viral vectors can be used in accordance with the present disclosure for delivery of a oligonucleotides, viral vectors suffer the drawback of potentially initiating undesired immune reactions. In addition, many viral vectors cause long term expression of gene, which is useful for some applications of gene therapy, but for applications where sustained gene expression is unnecessary or even undesired, transient transfection is a viable option. Viral vectors also involve insertional mutagenesis and genomic integration that can have undesired side effects. However, in accordance with one embodiment certain non-viral carriers, such as liposomes or exosomes can be used to deliver a miR-29 interference oligonucleotide to somatic cells in vivo.

TNT provides a method for localized gene delivery that causes direct transfection of tissues in vivo under immune surveillance without the need for any laboratory procedures. By using TNT with oligonucleotides or plasmids, it is possible to temporally and spatially control overexpression of a gene or inhibit expression of a target gene. Spatial control with TNT allows for transfection of a target area such as a portion of skin tissue without transfection of other tissues. Details regarding TNT devices have been described in US published patent application nos. 20190329014 and 20200115425, the disclosures of which are expressly incorporated by reference. Tissue nanotransfection allows for direct cytosolic delivery of cargo (e.g. , interference oligonucleotides or genes) into cells by applying a highly intense and focused electric field through arrayed nanochannels, which benignly nanoporates the juxtaposing tissue cell members, and electrophoretically drives cargo into the cells.

In accordance with one embodiment a pharmaceutical composition for enhancing wound closure is provided. In one embodiment the composition comprises an oligonucleotide at least 8 nucleotides in length, wherein the oligonucleotide has at least 80%, 85, 90%, 95% or 99% sequence identity to a continuous 8 nucleotide sequence of human mature miR-29a sequence (SEQ ID NO: 2), miR-29b (SEQ ID NO: 3), miR-29c sequence (SEQ ID NO: 4), SEQ ID NO: 5, SEQ ID NO: 6 or any complement of said sequences, and a pharmaceutically acceptable carrier. In one embodiment the oligonucleotide is an RNA comprising a locked nucleic acid. In one embodiment the siRNA of any of the embodiments disclosed herein comprise a locked nucleic acid at the N-terminal and/or C-terminal nucleotide in said oligonucleotide. In one embodiment the pharmaceutical compositions disclosed herein are used to promote wound healing in a subject, wherein an miR-29 inhibitor is transfected into the wound-edge tissue to reduce the function or activity of miR-29b and/or miR-29c, and thereby promoting wound healing.

### Example 1

### Wound Healing Pathway in Adult Skin Repair

Materials and Methods

Cell cultures used immortalized human keratinocytes (HaCaT) grown in Dulbecco's low-glucose modified Eagle's medium (Life Technologies, Gaithersburg MD). Human dermal microvascular endothelial cells (HMECs) were cultured in MCDB-131 medium (Life Technologies). Human skin fibroblast BJ cells (ATCC CRL-2522) were cultured in Eagle's Minimum Essential Medium (catalog no. 30-2003) per instructions. Cells were maintained in a standard culture incubator with humidified air containing 5% CO₂ and 10% FBS at 37° C. Unless otherwise noted, regular FBS was used for all experiments.

For transfection of miRNA mimics or miRNA inhibitors, DharmaFECT™ 1 transfection reagent was used to transfect HaCaT cells with miRIDIAN miR-29a, miR-29b and miR-29c mimic (50 nM), miR-29a, miR-29b and miR-29c hairpin inhibitor (100 nM) (Dharmacon) as described (Mol Ther 23, 1201-1210 (2015). Non-targeting miRNA mimic and inhibitors were transfected in the cells to serve as negative controls respectively. Cells were collected 48-72 h after transfection for further analysis as indicated.

miR-target 3′-UTR luciferase reporter assay was performed using HaCaT cells transfected with 100 ng pLuc-NPGPx-3′UTR plasmid (Origene) or a mutant (FIG. S4K) construct using Lipofectamine LTX/Plus reagent per manufacturer's protocol (28). The pLuc-NPGPx-3′UTR plasmid was designed based on the sequence of miR-29 binding sites and a total of 646 bp were cloned in the 3′ UTR of the pLuc-plasmid (NPGPx-3′UTR (SEQ ID NO: 8); mutated NPGPx-3′UTR (SEQ ID NO: 9). Data normalization was achieved by co-transfecting cells with Renilla plasmid. Cells were lysed after 24 h, and luciferase activity was determined using dual-luciferase reporter assay system (Promega). Data are presented as ratio of firefly to Renilla luciferase.

To prepare lipid-depleted serum, blood collected from human subjects was allowed to clot by addition of 250 IU thrombin (bovine origin) and 2.5 μl 1.25M CaCl₂ per 1 ml blood. The serum was collected, and lipid depletion was performed. Briefly, 1 ml serum was incubated overnight with 100 mg activated charcoal (Sigma) at 4° C. After centrifugation at 1200 g for 20 minutes, the supernatant was filtered (0.22 pm filter) and stored at −20° C.

Human wound biopsy samples were obtained from chronic wound patients at Ohio State University's Comprehensive Wound Center clinic. Blood was obtained from healthy adult consented human subject. All human studies were approved by OSU's Institutional Review Board (IRB). Declaration of Helsinki protocols was followed, and patients gave written informed consent. Experiments with human fetal skin samples were performed in Prof. Nilanjana Maulik's laboratory at the University of Connecticut Health, Farmington CT. Fetal skin samples were purchased from Advanced Bioscience Resources, Inc. California.

Male C57BL/6 mice (aged 8-10 weeks) were obtained from Harlan Laboratory. Mice homozygous (BKS.Cg-m^{+/+} Lepr^{db/J}, or db/db; stock no 000642) for spontaneous mutation of the leptin receptor (Lepr^{db}) or their respective non-diabetic lean control littermates m⁺/db (aged 10-12 weeks) that is an established model for impaired healing were obtained from Jackson Laboratory. Mutant mice carrying floxed Dicer1 (Dicer^{fl/fl} ) allele was a gift by Dr. Fuchs. Keratinocytes specific Dicer-ablated mouse (K14-Dicer^{-/-}) was generated by crossing Dicer^{fl/fl} mouse with mouse having Cre recombinase protein fused to estrogen-receptor ligand binding domain under keratin 14 promoter (STOCK Tg(KRT14-cre/ERT)20Efu/J; stock no:005107). The method for conditional deletion of dicer from keratinocytes was described previously (Mol Ther 23, 1201-1210 (2015). MCPIP1^{fl/fl} mice were a gift from Prof. Kolattukudy. Keratinocytes specific MCPIP1-ablated mouse (K14-MCPIP1^{-/-}) was generated by crossing MCPIP1^{fl/fl} mouse with mouse having Cre recombinase protein fused to estrogen-receptor ligand binding domain under keratin 14 promoter (STOCK Tg(KRT14-cre/ERT)20Efu/J; stock no:005107). Mice homozygous (B6.129S4-Ccl2^{tm1Rol}/J; stock no 004434) for spontaneous mutation of the MCP-1 were from Jackson Laboratory. Humanized CD34+ engrafted NOD.Cg-PrkdcscidIl2rgtm1Wjl (NSG) mice (age 24-28 weeks) from Jackson Laboratory (Bar Harbor, Maine) with stable engrafted human skin were used to test delivery efficiency of TNT2.0 using FAM-DNA. Female Yorkshire pig was used to test delivery efficiency of TNT2.0 in porcine skin.

All animal studies (mouse and pig) were performed in accord with protocols approved by OSU's Laboratory Animal Care and Use Committee and Indiana University's Laboratory Animal Resource Center. No statistical methods were used to predetermine sample size. Power analysis were not necessary. The animals were tagged and grouped randomly using a computer-based algorithm (www.random.org). No mice with the appropriate genotype were excluded.

For wounding, two 8×16 mm full-thickness excisional wounds were created on the dorsal skin, equidistant from the midline and adjacent to the 4 limbs. For lentivirus delivery, two 6-mm diameter full-thickness excisional wounds were developed on the dorsal skin of mice with a 6-mm disposable biopsy punch and splinted with a silicon sheet to prevent contraction thereby allowing wounds to heal through granulation and re-epithelialization. During the wounding procedure, mice were anesthetized by low-dose isoflurane (1.5%-2%) inhalation per standard recommendation. Each wound was digitally photographed at the time point indicated. Wound size was calculated by the ImageJ software.

Full-thickness dorsal fetal wounds were generated at E15.5 or E18.5 in FVB pregnant mice. After wounding, 1 μl phosphate-buffered saline containing 10% India ink was injected subcutaneously at the wound site. Skin from age-matched unwounded animals served as controls. All animal studies were approved by OSU's Institutional Animal Care and Use Committee (IACUC).

Animals were euthanized at the indicated time and wound edges were collected for analyses. For wound-edge harvest, 1-1.5 mm of tissue from the leading edge of the wounded skin was excised around the entire wound. Tissue was snap frozen and collected either in 4% paraformaldehyde or in optimal cutting temperature (OCT) compound.

Trans-epidermal water loss (TEWL) is as a reliable index to evaluate skin barrier function in vivo. TEWL was measured from the skin and wounds using DermaLab TEWL Probe (cyberDERM, Broomall PA). Data were expressed in g·m⁻²·h⁻¹.

In vivo dermal delivery of lentivirus was by intradermal injection of either ilenti (suppressing) or plenti (overexpressing) virus having different backbone. Briefly, ilenti-NPGPx (iLV-NPGPx) or plenti-NPGPx (pLV-NPGPx) with their respective control constructs (Applied Biological Materials) at titer 1×10⁷ cfu/mL (50 μL per wound) was intradermally injected into the skin 1 mm away from the wound edge 2 days before inducing wounds as described above. The injection procedure was repeated on the day of wounding and at day 3 post-wounding.

Tissue nanotransfection 2.0 in vivo TNT was performed as described previously with a modification in the chip design (Nat Nanotechnol 12, 974-979 (2017). The hollow microneedle array was fabricated on a double side polished silicon wafer using a standard semiconductor process in a cleanroom environment. First, the Si wafer was wet oxidized in a furnace at 1150° C. to grow 4 μm thermal oxide on both sides that served as a hard mask during the deep silicon etching. A 10 μm thick, positive photoresist of AZ 9260 was spin coated on one side of the silicon wafer followed by a prebake at 110° C. for 10 min. A direct laser writing system was used to expose a layout of 25 μm circle arrays followed by development in a diluted AZ400K solution to remove the exposed area. The 4 μm oxide was removed by a plasma etcher using CHF₃ chemistry. The wafer was then transferred to another plasma etching system to perform a deep Si etching called Bosch process, a common semiconductor process to achieve a vertical etching profile with a high-aspect ratio. After silicon etching of about 350-450 μm in depth to form the reservoir arrays, the wafer was flipped for the next step to etch the hollow microneedle arrays. A donut-shaped pattern was exposed onto the resist and the pattern was transferred to the oxide using the same set of steps as above. The wafer was then etched by the Bosch process until the hollow microneedles were connected to the reservoirs so that the cargo or the plasmid DNA fluid could freely flow from the reservoir to the hollow microchannel. SEM images showed the fabricated silicon hollow microneedle array having 170 μm length, 50 μm outer diameter, and 4 μm hollow diameter. When an electric pulse was applied between the TNT chip and tissue, the negatively charged plasma DNA traveled from the reservoir to nearby target cells by electrophoresis and entered them by electroporation. To test TNT2.0 delivery efficiency, FAM-DNA (5′/56-FAM/TACCGCTGCGACCCTCT-3′; SEQ ID NO: 7) was used in murine skin, porcine skin and human skin in humanized mouse.

Laser capture microdissection (LCM) used the PALM Technologies laser microdissection system (Bernreid, Germany) For epidermal LCM captures, sections were stained with hematoxylin for 30 s, subsequently washed with DEPC-H20 and dehydrated in ethanol. Epidermal fractions, identified based on histology, were typically cut and captured under a 20× ocular lens. Samples were catapulted into 25 μl of cell direct lysis extraction buffer (Invitrogen). About 15,00,000 μm² of tissue area was captured into each cap and the lysate was then stored at −80° C. for further processing.

RNA from cells or murine wound edge tissue samples was extracted using miRVana miRNA isolation kit (Ambion) per the manufacture's protocol. Specific TaqMan assays for miRs and the TaqMan miRNA reverse transcription kit were used to determine miR expression, followed by real time polymerase chain reaction (PCR) using the Universal PCR Master Mix (Applied Biosystems, Foster City CA). mRNA was quantified by real-time or quantitative (Q) PCR assay using the double-stranded DNA binding dye SYBR Green-I.

RNA samples (10 μg each) were used to detect the pre-miRNA-29c using miRNA Northern Blot Assay kit (Signosis, NB-0001) following the manufacturer's instructions. Northern blots were hybridized with biotin-labeled miR-29c probe (MF-0529) and pre- and mature miR-29c was detected based on difference in size.

Western blots was performed using antibodies against NPGPx (GeneTex; GTX108578, 1:2,000), MCPIP1 (GeneTex; GTX110807, 1:1,000), and signals were visualized using corresponding HRP-conjugated secondary antibody (Amersham, 1:3,000) and ECL Plus™ Western Blotting Detection Reagents (Amersham). GAPDH (Sigma-Aldrich; G9295, 1:15,000) served as loading control.

Immunohistochemistry (IHC) was performed as described previously (Mol Ther 25, 2502-2512 (2017). Immunostaining of NPGPx (GeneTex; GTX108578, 1: 400; GTX105683, 1:100 for human samples), MCPIP1 (GeneTex; GTX110807, 1:200), Collagen 3 (Abcam; ab7778, 1:100), Chondroitin sulfate (CS-56) (Abcam; ab11570, 1:200), HAS 3 (Novus Biologicals; NBP1-86328, 1:100), and Keratin14 (Covance; PRB-155P, 1:400) was performed on cryosections of wound sample using specific antibodies as described previously (The Journal of biological chemistry 282, 23482-23490 (2007)). Briefly, OCT embedded tissue were cryosectioned at 10 pm thick, fixed with cold acetone, blocked with 10% normal goat serum and incubated with specific antibodies against NPGPx (1:400), MCPIP1 (1:400), Keratin14 (1:400), overnight at 4° C. The signal was visualized by subsequent incubation with fluorescence-tagged appropriate secondary antibodies (FITC-tagged α-rat, 1:200; Alexa 488-tagged α-rabbit, 1:200; Alexa 568-tagged α-rabbit, 1:200) and counter stained with DAPI. Images were captured by microscope and the fluorescent intensity of images were quantified by software AxioVision Rel 4.6 (Carl Zeiss Microimaging). Paraffin-embedded sections were processed for picrosirius red and Masson trichome staining.

Statistical analysis used GraphPad Prism (GraphPad Software) v8.0. No statistical methods were used to predetermine sample size. The AACt value was used for statistical analysis of all RT-qPCR data. Statistical analysis between multiple groups were performed using one-way analysis of variance with the post hoc Sidak or Bonferroni multiple comparison test. Statistical analysis between two groups were performed using unpaired Student's two-sided t tests. P<0.05 was considered statistically significant. Significance levels and exact P values are indicated in all relevant figures. Data were assumed to be normally distributed for all analyses conducted. Data for independent experiments were presented as means±SEM unless otherwise stated.

### Results

FIG. 1 collectively demonstrates that NPGPx is wound inducible and abundant in developing fetal skin. Excisional wounding (8×16 mm) followed by tissue harvesting at time points 12 h, d1, d2, d3, d5, d7, d9, d11 and d14 was conducted in C57BL/6 (wild type) mice. FIG. 1A shows miR-29a, miR-29b and miR-29c transcript abundance in skin and wound-edge tissue of adult C57BL/6 mice at different time points post-wounding (PW) (n=4). FIG. 1C shows NPGPx transcript abundance in skin and wound-edge tissue in adult C57BL/6 mice (n=6). FIG. 1D shows Western blot analysis of NPGPx in skin and wound-edge tissue in adult C57BL/6 mice; GAPDH was the loading control and independent blots were repeated at least three times with similar results. FIG. 1E shows NPGPx transcript abundance in laser captured microdissected (LCM) keratinocytes from skin and day 7 wound-edge tissue of C57BL/6 mice (n=4). FIG. 1F shows Western blot analysis of NPGPx in murine fetal skin (E15.5-E18.5) and adult skin; GAPDH was the loading control and independent blots were repeated at least three times with similar results. FIG. 1G shows NPGPx transcript abundance in human fetal and adult skin (n=6, 5). FIG. 1H shows Western blot analysis of NPGPx in human fetal and adult skin; each lane indicates separate biological samples (n=5) and GAPDH was the loading control. All data were shown as mean±SEM. Data in FIGS. 1A and 1C were analyzed by one-way analysis of variance with the post-hoc Bonferroni multiple comparison test. Data in FIGS. 1E and 1G were analyzed by two-tailed unpaired Student's t test.

FIGS. 1A, 1C and 1D demonstrate the kinetics of NPGPx expression following wounding. In adult mice, excisional wounding (FIG. 1A), rapidly and transiently induced NPGPx expression at the wound-edge tissue (FIGS. 1C and 1D). Wound-inducible NPGPx was localized in the epidermis.

NPGPx expression was evaluated in murine fetal skin, since successful cutaneous wound healing recapitulates embryonic skin development in numerous aspects. There was copious expression of NPGPx in murine fetal skin on embryonic day (E) E15.5 (FIG. 1F). However, by E18.5, when murine fetal epidermal and dermal regenerative healing capacity is known to be lost, NPGPx expression was minimized and approached NPGPx levels in adult skin (FIG. 1F). In human fetal skin, NPGPx was also localized predominantly in epidermis and was more abundant than in adult epidermis. These data support that NPGPx is developmentally expressed in fetal keratinocytes, declines in expression in adult skin, but may be re-engaged following injury.

NPGPx was critical for fetal and adult wound closure and healing.

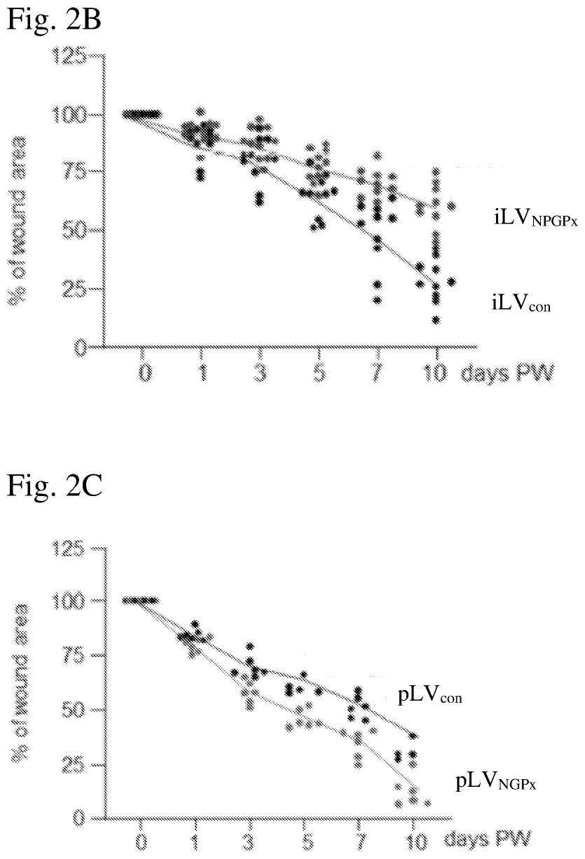

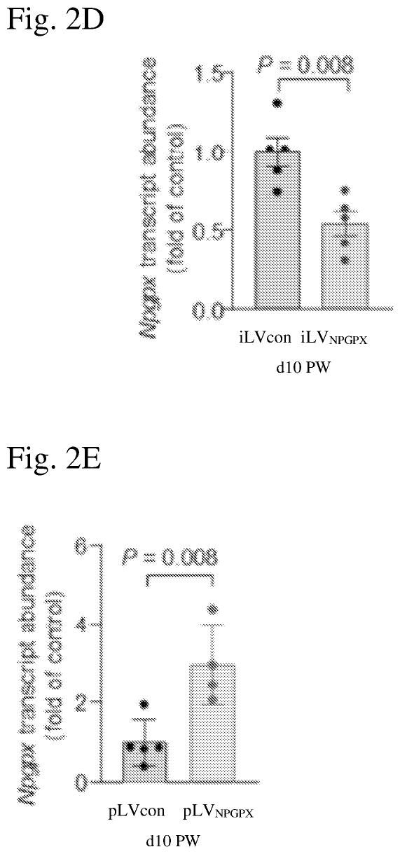

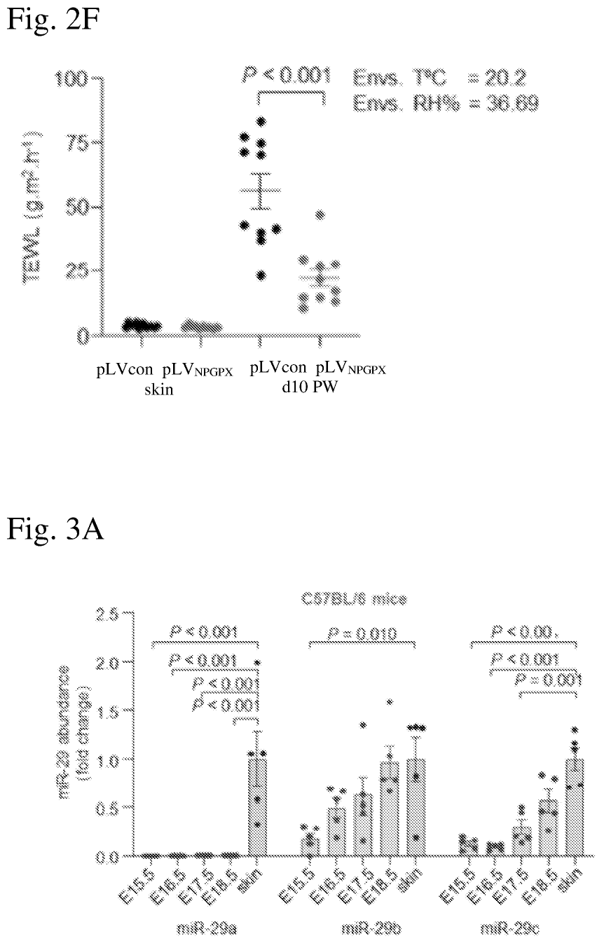

If NPGPx is involved in keratinocyte regeneration, then impairing NPGPx should impair wound re-epithelization. Indeed, in-utero delivery of sh-NPGPx lentiviral particles suppressing NPGPx (iLV-NPGPx) in E15.5 fetal skin, strikingly halted fetal wound healing with keratinocyte migration stalled at the wound edge. After in utero delivery of NPGPx suppressing lentivirus (iLV_{NPGPx}) or control lentivirus (iLV_{con}) in FVB mouse at E15.5, wound closure in the healing and non- healing wounds was measured on the day of presentation (d0) to wound clinic and as measured 30 days post-presentation (d30). Wounds with the greatest closure displayed highest NPGPx abundance (FIG. 2A). Digital photographs of excisional stented punch wound (6 mm) at different days and quantification by digital planimetry were taken following delivery of either iLV_{con} or iLV_{NPGPx} (FIG. 2B) or NPGP_{X} overexpressing lentivirus (pLV_{NPGPx}) with its respective control (pLV_{con}) (FIG. 2C) in C57BL/6 mice. (D: n=10; E: n=6). Delivery of pLV_{NPGPx} showed faster re-epithelialization compared to its respective control (pLV_{con}). All data were shown as mean±SEM.

NPGPx is critical for fetal and adult wound healing. FIGS. 2D and 2E show NPGPx transcript abundance at day 10 wound-edge tissue after either lentiviral suppression using iLV_{NPGPx} with its respective control iLV_{con} (FIG. 2D) or lentiviral overexpression using pLV_{NPGPx} with its respective control pLV_{con} (FIG. 2E) (FIG. 2D: n=5; FIG. 2E: n=5,4). Western blot analysis of NPGPx at day 10 wound-edge tissue after either lentiviral suppression using iLV_{NPGPx} with its respective control iLV_{con} or lentiviral overexpression using pLV_{NPGPx} with its respective control pLV_{con} showed similar results as the transcript abundance analysis.

Trans-epidermal water loss (TEWL) is as a reliable index to evaluate skin barrier function in vivo. FIG. 2F shows transepidermal water loss (TEWL) at d10 post-wounding in C57BL/6 mice treated with either pLVcon or pLVNPGPx (n=10).

These data demonstrate that NPGPx is pivotal in driving fetal wound regenerative re-epithelialization. NPGPx expression in healing and non-healing human skin wounds was examined. Wound biopsies from healing human cutaneous wounds had significantly more NPGPx expresses, compared to NPGPx expressed in either non-healing subjects (FIG. 2A) or in normal adult skin (FIGS. 1H). The NPGPx expressed in human epidermis correlated with the highest level of wound closure (FIG. 2A).

The significance of modulating epithelial NPGPx expression in adult murine wound closure was evaluated. Lentiviral particles were constructed to either suppress NPGPx abundance (iLV-NPGPx), or to overexpress NPGPx (pLV-NPGPx). These lentiviral particles were delivered intra-dermally into skin two days before cutaneous wounding. NPGPx suppression impaired adult wound closure (FIGS. 2B and 2D). NPGPx overexpression improved adult wound closure (FIGS. 2C and 2E). NPGPx overexpression accelerated wound re-epithelialization with significant improvement in skin barrier function (FIG. 2F); a critical functional test of re-epithelialization. NPGPx overexpression enhanced wound collagen deposition with increased levels of desirable collagen III, chondroitin sulfate (CS-56) and hyaluronic acid synthase 3; elevated expression of these three proteins is an established hallmark of regenerative fetal wound healing.

Augmenting NPGPx expression by gene delivery re-engaged elements of the robust re-epithelialization and regenerative tissue phenotype typical of fetal wound healing, to adult cutaneous wound healing.

Post-transcriptional regulation of epithelial NPGPx.

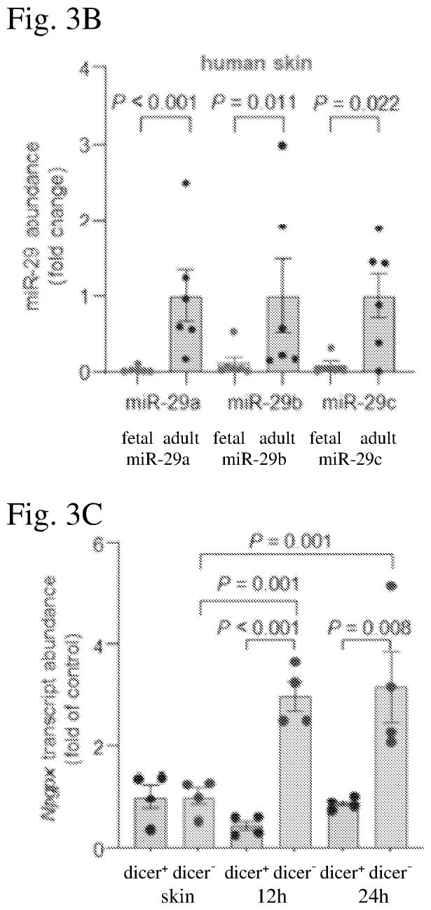

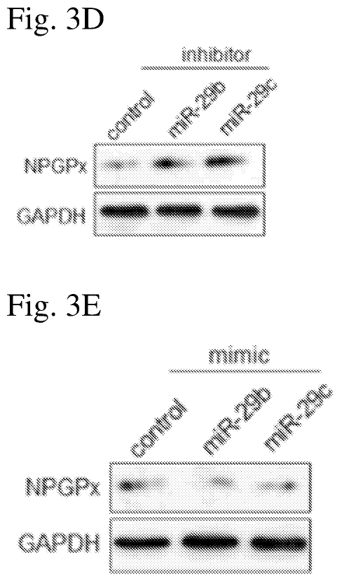

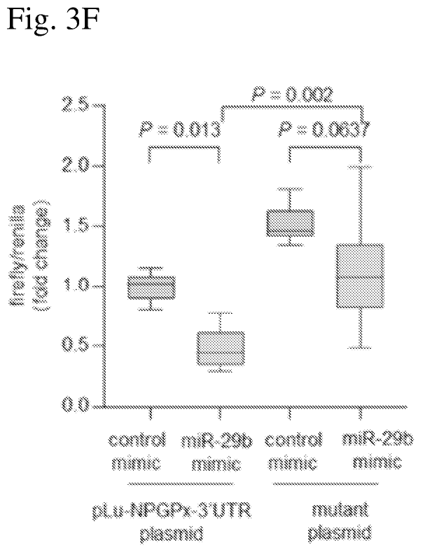

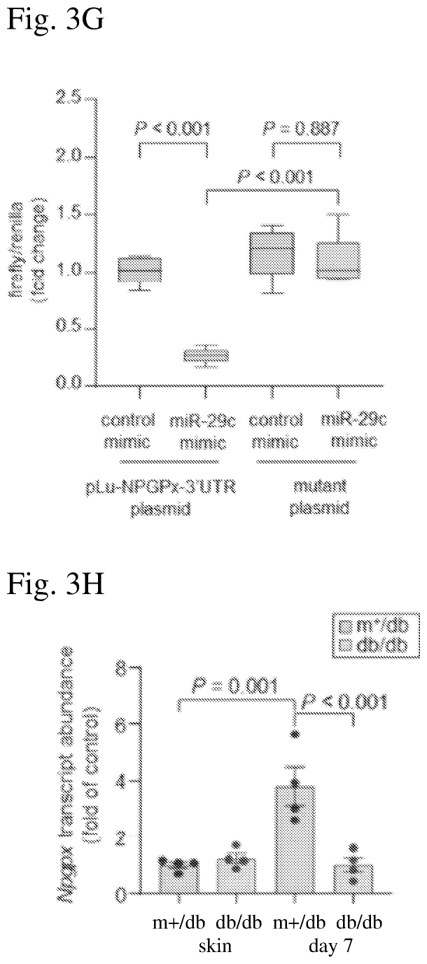

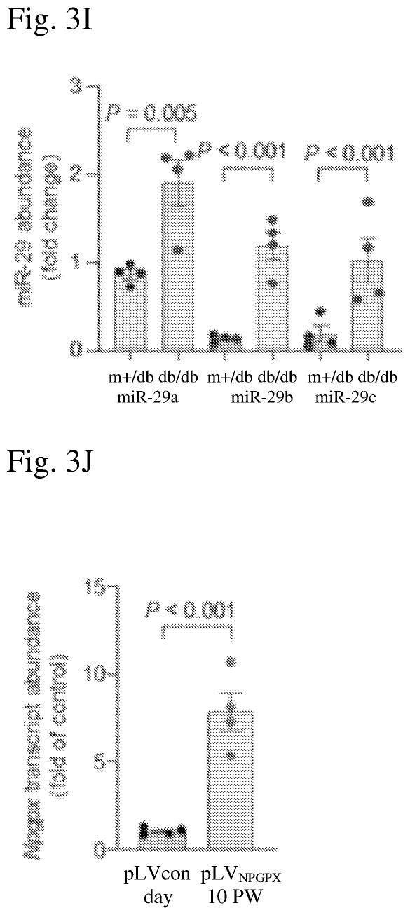

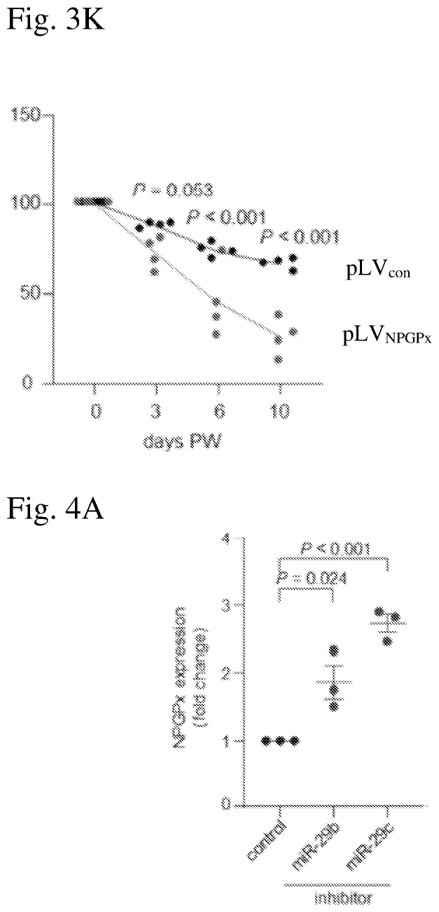

FIGS. 3A and 3B show miR-29a, miR-29b and miR-29c expression in fetal (E15.5-E-18.5) and adult skin of C57BL/6 mice (FIG. 3A), and fetal and adult human skin (FIG. 3B). (FIG. 3A: n=5; FIG. 3B: n=6, 6; 6,6; 5,6). FIG. 3C shows NPGPx transcript abundance in skin and wound-edge tissue in K14-dicer^{+/+} and K14-dicer^{-/-} mice (n=4).

Quantification of NPGPx intensity in wound-edge tissue (24h post-wounding) in K14-dicer^{+/+} and K14-dicer^{-/-} mice (n=3).

FIGS. 3D and 3E show Western blot analysis of HaCaT cells transfected with miR-29b or miR-29c inhibitor (FIG. 3D) and miR-29b or miR-29c mimic (FIG. 3E). GAPDH was the loading control. Independent blots were repeated at least three times with similar results. FIGS. 3F and 3G show miRNA target reporter luciferase assay in in HaCaT cells after delivery of miR-29b (FIG. 3F) and miR-29c (FIG. 3G) mimic (H: n=6; I: n=6). Data in FIGS. 3A, 3C, 3F and 3G were analyzed by one-way analysis of variance with the post-hoc Bonferroni multiple comparison test. Data in FIG. 3B were analyzed by two-tailed unpaired Student's t test

NPGPx gene delivery improved diabetic adult wound healing. FIG. 3H shows NPGPx transcript abundance in skin and day 7 wound-edge tissue of non-diabetic (m+/db, littermate control) and diabetic db/db mice (n=4). Western blot analysis of NPGPx from skin and day 7 wound-edge tissue from m+/db and db/db mice showed similar results as obtained for transcript abundance. Independent blots were repeated at least three times with similar results. FIG. 3I shows miR-29a, miR-29b and miR-29c expression in day 7 wound-edge tissue in m+/db and db/db mice (n=4). Fold change was calculated using respective skin (m+/db and db/db) as 1. FIG. 3J shows NPGPx transcript abundance of NPGPx in day 10 wound-edge tissue of diabetic db/db mice treated with either control (pLV_{con}) or NPGPx over expressing (pLV_{NPGPx}) lentivirus. Western blot analysis mice showed similar results as obtained for transcript abundance. GAPDH was used as loading control. Digital photographs of wounds in db/db mice were taken at day 0, 6 and 10 and wound closure was presented as percentage of initial wound area. (n=4) in FIG. 3K. All data were shown as mean±SEM. Data in FIG. 3H were analyzed by one-way analysis of variance with the post-hoc Sidak multiple comparison test. Data in FIGS. 3I and 3J were analyzed by two-tailed unpaired Student's t test.

Diabetes impairs skin wound closure.

Diabetic db/db mice with mutations of the leptin receptor display impaired wound healing and serve as a model for non-healing wounds. The wound-edge tissue of db/db mice was deficient in NPGPx expression (FIG. 3H), with concomitantly elevated transcript abundance of miR-29a, miR-29b and miR-29c at day 7 post-wounding (FIG. 3I). Correcting the NPGPx deficiency by overexpressing NPGPx in the wound edge tissue with a lentivirus (pLV-NPGPx) injected intra-dermally (FIG. 3J) markedly accelerated wound closure in db/db mice. Thus, augmenting NPGPx expression at a wound site may overcome the deleterious effects of diabetes on wound closure, with associated blunting of NPGPx expression in keratinocytes (FIG. 3K).

There is post-transcriptional regulation of epithelial NPGPx.

In murine fetal skin, the expression pattern of the miR-29 family was low from E15.5 until E17.5 (FIG. 3A), after which it gradually increased. This expression pattern was an exact inverse of the NPGPx expression pattern in the epidermis (FIGS. 1F and 1G). In human fetal skin, there was a similar expression pattern (FIG. 3B). If miR-29 targets NPGPx transcripts for degradation, then there should be significantly increased NPGPx expression under conditions of low miR transcript abundance following tissue injury.

Excisional wounds were developed in K14 (keratinocyte restricted) conditional dicer-ablated mice; animals in which all miR would be diminished in keratinocytes. In these mice, wound inducible NPGPx transcripts in keratinocytes appeared earlier at 12 h (FIG. 3C). NPGPx protein expression was also elevated at 24 h post-wounding. These results are consistent with miR-29 regulating NPGPx. The specific role of miR-29 family members in regulating NPGPx expression was determined to demonstrate a direct role for miR-29 in regulating NPGPx expression. The miR-29 family members share the same sequence homology at the predicted binding site (seed region) that decides the fate of coding gene miRNA abundance. Unlike miR-29b and miR-29c, transfection of miR-29a mimic and inhibitor to adult human keratinocytes did not change expression of NPGPx.

miR-29b and miR-29c was evaluated determine whether NPGPx was a direct target for the miR-29 family members. Adult human keratinocytes were transfected with mimics and inhibitors of miR-29b and miR-29c. Suppression of miR-29b and miR-29c significantly elevated NPGPx expression (FIG. 4B). Delivery of miR-29b and miR-29c mimics significantly suppressed both NPGPx expression (FIG. 4B), as well as NPGPx 3′-UTR reporter luciferase activity. FIG. 4C gives the sequence of wild-type NPGPx 3′-UTR (top; SEQ ID NO: 8) and NPGPx 3′-UTR (SEQ ID NO: 9) with the mutation of predicted binding site (seed region) cloned in the reporter construct (bottom). The top 10 predicted miR-29a-c binding sites were marked with bold text. Positions mutated were marked with underlined text. Mutations of the predicted binding sites (seed sequences) in the 3′-UTR of NPGPx (FIG. 4C) abolished miR-29b and miR-29c dependent translational repression. This established that miR-29b and miR-29c specifically bind to the predicted sites to enact post-transcriptional gene silencing of NPGPx. Thus, NPGPx was established as a direct target of miR-29b and miR-29c. Elevated NPGPx abundance in E15.5-17.5 fetal skin was reconciled with low miR-29b and miR-29c abundance and established a causal relationship between low miR-29b and miR-29c abundance in a tissue with concomitant up-regulation of NPGPx expression.

Lentiviral particles delivered anti-miR-29b and anti-miR29c oligonucleotides into the wound edge tissue of diabetic db/db mice, known to display low NPGPx but high miR-29b and miR-29c abundance at wound edge tissue (FIGS. 3H and 3I). Wound closure occurred significantly more quickly (FIG. 5A) by induction of NPGPx expression in the skin following miR-29b and miR-29c suppression (FIG. 5B). Thus, augmenting NPGPx expression in the epidermis mediates wound closure in diabetic mice and provides a mechanism to induce NPGPx expression by miR-29b and miR-29c suppression.

Wound inducible endoribonuclease MCPIP1 causes miR 29c suppression.

FIG. 4D shows MCPIP1 transcript abundance in skin and wound-edge tissue at different time points PW in adult C57BL/6 mice, (n=5, 4). Digital photographs of wounds in K14-MCPIP1^{+/+} and K14-MCPIP1^{-/-} mice were taken and wound closure presented as percentage of initial wound area (n=6); scale, 2 mm (FIG. 4E). FIG. 4F shows transepidermal water loss (TEWL) at d12 post-wounding is lower in K14-MCPIP1^{+/+} and higher in K14-MCPIP1^{-/-} mice suggesting poor barrier function, (n=6). Expression of miR-29b and miR-29c was elevated in wound-edge laser captured keratinocytes from K14-MCPIP1^{+/+} and K14-MCPIP1^{-/-} mice. (n=5, 6). In summary, the data is consistent with increased MCPIP1 concentrations resulting in decreased pre-miRNA concentrations and decreased miR-29b and miR-29c levels which result in increased concentrations of NPGPx concentrations which result in faster wound healing.

All reporter construct were cloned in pMirTarget vector by Origene. All data were shown as mean±SEM. Data were analyzed by two-tailed unpaired Student's t test.

Use of topical tissue nanotransfection (TNT) chip 2.0 to deliver agents to demonstrate increased NPGPx and decreased miR-29b and miR-29c result in increased wound healing.

TNT of LNA-control, LNA-anti-miR-29b, and LNA-anti-miR-29c was performed in K14-MCPIP1^{-/-} mice at day 1 post-wounding. Wound closure is presented as percentage of initial wound area. Scale, 2mm. (n=6,7,7). FIGS. 5C and 5D show expression of miR-29b (FIG. 5C) and miR-29c (FIG. 5D) in laser-captured epidermis of C57BL/6 mice 24h post-TNT of LNA-control, LNA-anti-miR-29b, and LNA-anti-miR-29c mice (n=4). FIG. 5E presents wound closure data (FIG. 5F) at day 12 wounds in K14-MCPIP1^{-/-} mice based on percent wound closure, showing accelerated re-epithelialization in wounds treated with LNA-anti-miR-29b, and LNA-anti-miR-29c relative to LNA-control. All data were shown as mean±SEM. Data in FIG. 5C and FIG. 5D were analyzed by two-tailed unpaired Student's t test. Data in FIG. 5E was analyzed by one-way analysis of variance with the post-hoc Bonferroni multiple comparison test.

Suppression of miR-29b and miR-29c by lentivirus (iLVmiR-29b and iLVmiR-29c) delivery in db/db mice induced NPGPx expression and improved diabetic adult wound healing. Diabetic db/db mice treated with control (iLV_{con}), miR-29b and miR-29c suppressing lentivirus (iLV_{miR-29b} and iLV_{miR-29c}) at day 12 show accelerated re-epithelialization in wounds relative to control (FIG. 5F). Wound closure is presented as percentage of initial wound area. (n=8,8,6). Scale, 2mm. FIG. 5G shows the expression of NPGPx in db/db mice treated with either iLV_{con}, iLV_{miR-29b} or iLV_{miR-29c} at day 12 post wounding. (n=4). The concentrations of detected NPGPx correlated with the speed of wound closure as shown in FIG. 5F. All data were shown as mean±SEM. Data were analyzed by one-way analysis of variance with the post-hoc Bonferroni multiple comparison test.

Cutaneous delivery of reprogramming molecules by TNT was efficient and effective in directly reprogramming dermal fibroblast cells into a variety of functionally distinct lineages. Modified TNT silicon chips (TNT2.0) have longer needle height of 170 μm and pore diameter of 4 μm. Modifying the electrical charge applied to the chip and skin led to step-wise increases in the depth of transfection of fluorescein amidite (FAM)-labeled DNA in mice and dermal delivery in pig skin and human skin engrafted in NSG mice. Delivery of LNA-anti-miR-29b and anti-miR-29c by TNT to the skin of wound healing deficient K14-MCPIP1^{-/-} mice led to a significant increase in wound closure (FIG. 5A) with a robust increase in expression of NPGPx in wound edge keratinocytes as expected following miR-29b and miR-29c suppression post-TNT application (FIGS. 5C and 5D).

MCPIP1 is induced post-wounding independent of MCP1.

FIG. 6A shows Western blot analysis and FIG. 6B shows quantification of MCPIP1 from skin and wound-edge tissue collected at 6 h post-wounding (PW) from wild type (WT) and MCP-1 knockout mice. GAPDH was a loading control. Independent blots were repeated at least three times with similar results. Data are expressed as mean±SEM (n=3). FIG. 6C shows the role of serum in the induction of MCPIP1 and the expression of MCPIP1 in primary human keratinocytes after removal of bioactive phospholipids from serum by charcoal stripping method (n=4). As shown by the data, the presence of a blood clot and MCP-1 increases cellular concentrations of MCPIP1. Wound-edge tissue analysis identified a transient downregulation of pre-miR-29c transcripts at 12 h post-wounding, followed by lowering of mature miR-29c levels 1-2 days later. This identified a plausible mechanism to rapidly diminish the abundance of miR-29c in post-wound tissue. Wound-induced changes in endoribonuclease expression could degrade pre-miR-29c abundance.

MCPIP1 (Zc3h12a) is an early inducible endoribonuclease that could degrade pre-miR-29c. MCPIP1 cleaves the terminal loops of pre-miRNAs and counteracts Dicer, leading to de novo miRNA biosynthesis inhibition. MCPIP1 was induced in wound-edge keratinocytes and in cells within the blood clot at 6 h post-wounding (FIG. 4D) to levels higher than those in fetal skin post-wounding. This may account for degradation of wound-edge pre-miR-29c at 12 h post wounding. MCPIP1 was not expressed in E15.5 or E18.5 fetal skin or wound edge tissue or in normal human skin, implying that adult cutaneous wounding activates a specific wound inducing pathway.

A keratinocyte-specific tamoxifen inducible transgenic mouse, to delete MCPIP1 expression in a restricted fashion, was generated to examine the role of MCPIP1 in regulating miR-29b and miR-29c abundance in keratinocytes. Wound closure was markedly stalled in K14-MCPIP1^{-/-} mice lacking MCPIP1 expression in keratinocytes (FIG. 4E). K14-MCPIP1^{-/-} mice displayed diminished NPGPx expression but concomitantly elevated abundance of miR-29b and miR-29c in the wound edge tissue. This impairment in wound healing was evident by poorer barrier function (FIG. 4F).

These data establish a critical role of MCPIP1 expression in keratinocytes in regulating the abundance of miR-29b and miR-29c that directly modulates NPGPx abundance and wound re-epithelialization.

Monocyte chemoattractant protein-1 (MCP-1) is the classical inducer of MCPIP1 and may be a wound-inducible regulator of MCPIP1 abundance. Wound-inducible MCPIP1 was evident, though at a lower level of abundance, in MCP-1 null mice (FIGS. 6A and 6B). Other inducers of MCPIP1 may be relevant to the adult wound microenvironment.

In cell cultures, experimental human blood clots induced MCPIP1 in keratinocytes, but not in endothelial or fibroblast cells. Post-clot human serum was active in inducing MCPIP1 in keratinocytes. Removal of all serum lipids by charcoal stripping abrogated the induction of MCPIP1 to control serum albumin levels (FIG. 6C). These results suggested that serum lipid components within the blood clot may play a role, along with MCP1 in inducing MCPIP1 expression at the wound edge post-injury.

Suppression of miR-29b and miR-29c rescues wound healing in K14-MCPIP1 knock out mice.

Given the role of miR-29b and miR-29c abundance in fetal wound regenerative healing and in post-injury wound healing in adult skin, topical tissue nanotransfection (TNT) to deliver locked nucleic acid (LNA) anti-miRs to the wound edge in K14-MCPIP1^{-/-} mice may rescue the delayed wound healing typically displayed by these animals. Topical delivery of anti-miR oligonucleotides by TNT is a direct approach to inducing NPGPx in skin keratinocytes that may be beneficial in human subjects whose non-healing wounds lack NPGPx.

Fetal murine and fetal human skin tissue is rich in NPGPx. Adult tissue is not rich in NPGPx. Fetal murine skin re-epithelialization post-injury requires NPGPx. Adult human cutaneous wounds that heal express high levels of NPGPx. Adult human cutaneous wounds that do not heal do not express NPGPx. Diabetes in adult mice blunts NPGPx expression at the edge of induced wounds and the wounds heal slowly.

Augmenting NPGPx expression plays a direct role in accelerating wound closure in diabetic adult mice cutaneous wounds by enhanced re-epithelialization. NPGPx has an important role in fetal and adult skin biology: NPGPx is a catalytically silent GPx family member that augments cutaneous wound re-epithelialization and closure. Results from murine studies are anticipated to translate to benefit humans, particularly diabetic patients. The rescue of diabetic murine wound healing by modulating the pathway:

↓miR29c→↑NPGPx
permits therapeutic intervention by replenishing key missing links of the healing process.

Key elements of the fetal repair path exist in the adult wound tissue. These elements are inherently purposed to promote adult repair by a pathway required for fetal skin repair. Execution of this physiologic wound repair mechanism is conflicted by adult conditions, resulting in poorer quality repair in healthy adults and impaired repair in diabetics.

Therapeutic bolstering of the NPGPx pathway in adult wounds, including the diabetic state, will more successfully engage the fetal regenerative repair pathway delivering improved healing outcomes. Use of a topical TNT approach to augment the physiological mechanisms for re-engaging NPGPx expression in wounds presents an imminently translational (feasibility to deliver through pig and human skin demonstrated), non-viral, minimally invasive, and widely applicable device for therapeutic intervention.

1. A method of accelerating wound closure in adult skin of a subject, said method comprising the step of increasing the concentration of the protein nonselenocysteine-containing phospholipid hydroperoxide glutathione peroxidase (NPGPx) in the cells of wound-edge tissue.

2. The method of claim 1 wherein the wound to be treated is a chronic wound in a diabetic patient.

3. The method of claim 1 wherein wound-edge tissue is transfected with a modifier of miR-29 activity in an amount effective to lower miR-29 activity and increase NPGPx expression, said modifier of miR-29 activity is
i) an oligonucleotide at least 6 nucleotides in length, wherein the oligonucleotide has at least 95% sequence identity to a continuous nucleotide complementary sequence of SEQ ID NO: 11.;
ii) an oligonucleotide at least 6 nucleotides in length, wherein the oligonucleotide has at least 95% sequence identity to a continuous nucleotide sequence of human mature miR-29a (SEQ ID NO: 2), miR-29b (SEQ ID NO: 3) or miR-29c (SEQ ID NO: 4) or a complement of SEQ ID NO: 2, SEQ ID NO: 3 or SEQ ID NO:4, thereof; or
iii) an oligonucleotide comprising the sequence UGGUGCU (SEQ ID NO: 5).

4. The method of claim 1 wherein the modifier the of miR-29 activity is a gene encoding the protein endoribonuclease monocyte chemoattractant protein-induced protein 1 (MCPIP1).

5-7. (canceled)

8. The method of claim 3 wherein said oligonucleotide is an RNA comprising a locked nucleic acid at the N-terminal and/or C-terminal nucleotide in said oligonucleotide.

9. (canceled)

10. (canceled)

11. The method of claim 3 wherein said oligonucleotide is delivered into the cytosol of human epidermal and dermal cells, optionally into the cytosol of keratinocyte cells, wherein
i) the oligonucleotide is delivered into the cytosol of cells via skin electroporation or tissue nanotransfection; or
ii) the oligonucleotide is delivered into the cytosol of cells via a viral vector or lipid vesicle.

12. (canceled)

13. (canceled)

14. A pharmaceutical composition for enhancing wound closure, said composition comprising an oligonucleotide at least 6 nucleotides in length, wherein the oligonucleotide has at least 85% sequence identity to a continuous complimentary nucleic acid sequence of
i) human mature miR-29a (SEQ ID NO: 2);
ii) human mature mir29b (SEQ ID NO: 3); or
iii) human mature mir29c (SEQ ID NO: 4); and
a pharmaceutically acceptable carrier.

15. The composition of claim 14 wherein said composition comprises a mixture of different oligonucleotides selected from any combination of i), ii), and iii).

16. The composition of claim 14 wherein said oligonucleotide comprises the sequence UGGUGCU (SEQ ID NO: 5).

17. The composition of claim 14 wherein said oligonucleotide is an RNA comprising a locked nucleic acid at the N-terminal and/or C-terminal nucleotide in said oligonucleotide.

18. (canceled) . 19. (Canceled).

20. A method to promote wound healing in a subject, said method comprising the step of administering an miR-29 inhibitor to a wound on subject.

21. The method of claim 20 wherein the miR-29 inhibitor is transfected into the wound-edge tissue to reduce the function or activity of miR-29b and/or miR-29c, and thereby promoting wound healing.

22. The method of claim 20, where the miR-29 inhibitor is an oligonucleotide having at least 95% sequence identity to a continuous 8 nucleotide complimentary sequence of human mature miR-29 sequence selected from the group consisting of SEQ ID NO: 2, SEQ ID NO: 3 and SEQ ID NO: 4) or a complement thereof.

23. The method of 22 wherein said oligonucleotide comprises the sequence UGGUGCU (SEQ ID NO: 5).

24. The method of 22 wherein the subject is a diabetic patient.

25. A method to accelerate wound healing in adults comprising topically delivering the composition of claim 14, by topical tissue nanotransfection (TNT) under conditions sufficient to increase nonselenocysteine-containing phospholipid hydroperoxide glutathione peroxidase (NPGPx) activity in skin keratinocytes and result in wound healing.

26. The method of claim 25 wherein an oligonucleotide having at least 95% sequence identity to a continuous 8 nucleotide sequence of human mature miR-29 sequence (SEQ ID NO: 4) or a complement thereof, is transfected into the cells of wound-edge tissues.


SEQUENCE LISTING (11 sequences)

SEQ ID NO: 1 - RNA, 23 nt, Homo sapiens
cuccgcaaacucaaguggugcuu

SEQ ID NO: 2 - RNA, 22 nt, Homo sapiens
uagcaccaucugaaaucgguua

SEQ ID NO: 3 - RNA, 22 nt, Homo sapiens
uagcaccauuugaaaucauguu

SEQ ID NO: 4 - RNA, 22 nt, Homo sapiens
uagcaccauuugaaaucgguua

SEQ ID NO: 5 - RNA, 7 nt, Homo sapiens
agcacca

SEQ ID NO: 6 - RNA, 8 nt, Homo sapiens
cgauuuca

SEQ ID NO: 7 - DNA, 17 bp, Homo sapiens
taccgctgcgaccctct

SEQ ID NO: 8 - DNA, 626 bp, Homo sapiens
ccaccgcgtctcctcctccaccacctcatcccgcccacctgtgtggggctgaccaatgcaaactcaaatggtgcttcaaagggagagacccactgactctccttcctttactcttatgccattggtcccatcattcttgtgggggaaaaattctagtattttgattatttgaatcttacagcaacaaataggaactcctggccaatgagagctcttgaccagtgaatcaccagccgatacgaacgtcttgccaacaaaaatgtgtggcaaatagaagtatatcaagcaataatctcccacccaaggcttctgtaaactgggaccaatgattacctcatagggctgttgtgaggattaggatgaaatacctgtgaaagtgcctaggcagtgccagccaaataggaggcattcaatgaacattttttgcatataaaccaaaaaataacttgttatcaataaaaacttgcatccaacatgaatttccagccgatgataatccaggccaaaggtttagttgttgttatttcctctgtattattttcttcattacaaaagaaatgcaagttcattgtaacaatccaaacaatacctcacgatataaaataaaaatgaaagtatcctcctca

SEQ ID NO: 9 - DNA, 626 bp, Homo sapiens
ccaccgcgtctcctcctccaccacctcatcccgcccacctgtgtggggcactggatagcaaacagtttaccacgatcaaagggagagacccactgactctccttcctttactcttatgcctaaccacccatcaaagttcaccgggaaaaattctagtattttgattatttgaatcttacagcaacaaataggaactcctggccaatgagagctcttgaccagtgaatcacctcggctaacgaacgagttgccaacaatttactcagcgaaatagaagtatatcaagcaataatctcccacccaaggcttctgtaaactggctggaatctaaaccagttacccgacttgtgaggattaggatgaaattggtcagattcaccctagcgagtcggagcgtttaaccagcgattcaatgaacattttttgcatataaaccaaaaaataacttgttatcaataaaaacttgcatccaacatgaatttcctcggctagataatccaggcgtttccattacaacttgttatttcctctgtattattttcttcattacaaaagaaatgcaagttcattgtaacaatccaaacaatacctcacgatataaaataaaaatgaaagtatcctcctca

SEQ ID NO: 10 - RNA, 64 nt, Homo sapiens
augacugauuucuuuugguguucagagucaauauaauuuucuagcaccaucugaaaucgguuau

SEQ ID NO: 11 - RNA, 64 nt, Homo sapiens
auaaccgauuucagauggugcuagaaaauuauauugacucugaacaccaaaagaaaucagucau